(19) United States
(12) Patent Application Publication
(10) Pub. No.: US 2002/0182645 A1
(43) Pub. Date: Dec. 5, 2002

(54) Direct selection of cells by secretion product

(76) Inventors: Stefan Miltenyi, Bergisch Gladbach (DE); Andreas Radbruch, Bonn (DE); Rudi Manz, Koln-Sulz (DE)

Correspondence Address: Randolph Ted Apple, Morrison & Foerster LLP, 755 Page Mill Road, Palo Alto, CA, 94304, US

(21) Appl. No.: 10/094,461

(22) Filed: Mar. 7, 2002

Related U.S. Application Data
(63) Continuation of application No. 08/441,259, filed on May 15, 1995, now abandoned.

(51) Int. Cl. G01N 33/53; G01N 33/567; C12N 5/00

(52) U.S. Cl. 435/7.2; 435/325

(57) ABSTRACT

Cells can be labeled with products which they secrete and release in an efficient manner by coupling the cells at their surface to a specific binding partner for the product and allowing the product to be captured by the specific binding partner as it is secreted and released. The product-labeled cells can then be further coupled to suitable labels if desired and separated according to the presence, absence or amount of product.

### TECHNICAL FIELD

The invention is in the field of analysis of cell populations and cell separation. More particularly, the invention concerns analysis and separation techniques based on primary labeling of cells with their secreted products through capture of these products by a specific binding partner for the product anchored to the cell surface.

### BACKGROUND ART

Numerous attempts have been made to analyze populations of cells and to separate cells based on the products which they produce. Such approaches to cell analysis and separation are especially useful in assessing those cells which are capable of secreting a desired product, or which are relatively high secretors of the product. Prior art methods include cloning in microtiter plates and analysis of the culture supernatant for secreted product, cloning in agar and analysis by methods for identification of the secreted product of the localized cells; the identification methods include, for example, Plaque assays and western blotting. Most methods for analysis and selection of cells based upon product secretion use the concept of physical isolation of the cell, followed by incubation under conditions that allow product secretion, and screening of the cell locations to detect the cell or cell clones that produce the secreted product. For cells, in suspension, after the cells have secreted the product, the product diffuses from the cell without leaving a marker to allow identification of the cell from which it was secreted. Thus, secretor cells cannot be separated from non-secretor cells with this system.

In other cases, both secretor and non-secretor cells may associate the designated product with the cell membrane. An example of this type of system are B-cell derived cell lines producing monoclonal antibodies. It has been reported that these types of cell lines were separated by Fluorescence actived cell sorting (FACS) and other methods reliant upon the presence of antibody cell surface markers. However, procedures that analyze and separate cells by markers that are naturally associated with the cell surface may not accurately identify and/or be used in the separation of secretor cells from non-secretor cells. In addition, systems such as these are not useful in identifying quantitative differences in secretor cells (i.e., low level secretors from high level secretors).

A method that has been used to overcome the problems associated with product diffusion from the cells has been to place the cell in a medium which inhibits the rate of diffusion from the cell. A typical method has been immobilize the cell in a gel-like medium (agar), and then to screen the agar plates for product production using a system reliant upon blotting, for example Western blots. These systems are cumbersome ane expensive if large numbers of cells are to be analyzed for properties of secretion, non-secretion, or amount of secretion.

Kohler et al. have described a system in which mutants of a hybridoma line secreting IgM with anti-trinitrophenyl (anti-TNP) specificity were enriched by coupling the hapten to the cell surface and incubating the cells in the presence of complement. In this way, cells secreting wild-type Ig committed suicide, whereas cells secreting IgM with reduced lytic activity preferentially survived. Kohler and Schulman, Eur. J. Immunol. 10:467-476 (1980).

Other known systems allow the cells to secrete their products in the context of microdroplets of agarose gel which contains beads that bind the secretion products, and encapsulation of the cells. Such methods have been disclosed in publications by Nir et al., Applied and Environ. Microbiol. 56: 2870-2875 (1990) and Nir et al., Applied and Environ. Microbiol. 56:3861-3866 (1991). These methods are unsatisfactory for a variety of reasons. In the process of microencapsulation statistical trapping of numbers of cells in the capsules occurs, resulting in either a high number of empty capsules when encapsulation occurs at low cell concentrations, or multiple cells per capsule when encapsulation occurs at high cell concentrations. In order to analyze and separate single cells or single cell clusters by this technique, large volumes must be handled to work with relatively small numbers of cells because of the numbers of empty capsules and because of the size of the microcapsules (50-100 μm). The large volume of droplets results in background problems using flow cytometry analysis and separation. In addition, the capsules do not allow separation using magnetic beads or panning for cell separation.

Various methods have been used to couple labels to cell surfaces where the label is intended for direct detection, such as a fluorochrome. For example, hydrophobic linkers inserted into the cell membrane have been used to couple fluorescent labels to cells have been described in PCT WO 90/02334, published Mar. 8, 1990. Antibodies directed to HLA have also been used to bind labels to cell surfaces. Such binding results in a smaller dimension than the encapsulated droplets described above and such cells can conveniently be used in standard separation procedures including flow cytometry and magnetic separations.

It has now been found that by anchoring a specific binding agent into the cell surface using an appropriate coupling mechanism, secreted products of the cells can be captured and cells sorted on the basis of the presence, absence or amount of product.

### DISCLOSURE OF THE INVENTION

The invention provides a method for convenient analysis and cell separation based on the products secreted by the cells. The cells are provided with a capture mechanism for the product, which can then be used directly as a label in some instances, or the bound product can be further labeled via structures that bind specifically, to the product and that are labeled with traditional labeling materials such as fluorophores, radioactive isotopes, chromophores or magnetic particles. The labeled cells are then separated using standard cell sorting techniques based on these labels. Such techniques include flow cytometry, magnetic gradient separation, centrifugation, and the like.

Thus, in one aspect, the invention encompasses a method to separate cells according to a product secreted and released by the cells, which method comprises effecting a separation of cells according to the degree to which they are labeled with said product, wherein labeling with the product is achieved by coupling the surface of the cells to a specific binding partner for the product and culturing the cells under conditions wherein the product is secreted, released and specifically bound to said specific binding partner, and wherein the labeled cells are not lysed as part of the separation procedure.

Another aspect of the invention is a composition of matter which comprises cells capable of capturing a product secreted and released by the cells wherein the surface of the cells is coupled to a specific binding partner for the product.

Still another aspect of the invention is cells and progeny thereof separated by the above-described method.

Yet another aspect of the invention is a method to label cells with a product secreted and released by the cells, which method comprises coupling the surface of the cells to a specific binding partner for the product, and culturing the cells under conditions wherein the product is secreted and released.

An additional aspect of the invention is a method of analyzing a population of cells to determine the proportion of cells that secrete a varying amount of product relative to other cells in the population, the method comprising labeling the cells by the above-described method, further labeling the cells with a second label that does not label the captured product, and detecting the amount of product label relative to the second cell label.

Still another aspect of the invention is a kit for use in the dectection of cells that secrete a desired product, the kit comprising: a material for use in preparing gelatinous cell culture medium, said medium to be used for cell incubation for the production of the desired secreted product; a product capture system comprised of anchor and capture moieties; a label for detecting the captured product; and instructions for use of the reagents, all packaged in appropriate containers.

### BRIEF DESCRIPTION OF THE DRAWINGS

FIG. 1 is a scheme for the introduction of biotinyl and palmitoyl groups onto Dextran.

FIG. 2 is a scheme for the reaction of N-hydroxysuccinimide esters with primary amino groups in basic form.

FIG. 3a and FIG. 3b, respectively, are photocopies of traces of the FACScans of binding of streptavidin to cells treated with biotinyldextran and biotinylpalmitoyldextran.

FIG. 4 are photocopies of traces of the FACScan results: a) unbiotinylated cells treated with streptavidin-FITC (negative control); b) cells treated with biotinylpalmitoyldextran and then with streptavidin-FITC; c) cells incubated with biotinyl-anti-αβ₂m and treated with streptavidin-FITC.

FIG. 5 is a graph showing the titration of the binding of IgM to cells carrying conjugates of biotinylpalmitoyldextran and entrapment antibodies.

FIG. 6 are photocopies of traces of FACScan results of the entrapment with time of IgM on cells carrying entrapment antibody avidin-biotin conjugates. Panels (a), (b), (c), and (d) are the traces at 10 min, 30 min, 1 h, and 2 h, respectively.

FIG. 7 are photocopies of traces of FACScan showing the effect of the concentration of methylcellulose in the medium on entrapment. FIGS. 7a and 7b show the entrapment of antibodies by cells incubated in 2.5% and 1% methylcellulose medium, respectively.

FIG. 8 is a FACScan representation of stained cells before and after separation based on entrapment of secreted antibodies in 2.5% methylcellulose containing medium. The cells are shown in the FIG. 8a before separation. FIG. 8b shows the negative fraction after the separation. FIG. 8c shows the positive fraction after the separation.

FIG. 9a are photocopies of traces of FACScan results showing the effect of different added substances in the culture medium during the secretion phase. FIGS. 9a, 9b, and 9c show the capture of product by anti-product antibodies on cells when the cells are incubated in culture medium, culture medium with 40% BSA, and culture medium with 20% BSA plus 20% gelatin, respectively.

FIGS. 10 and 11 are FACScan representations of stained cells after labeling with capture antibody (10a, 11a), after the secretion phase (10b, 11b), and after magnetic separation, wherein (10c, 11c) are the magnetic fraction, and (10d and 11d) are the nonmagnetic fraction.

### MODES OF CARRYING OUT THE INVENTION

The invention employs a mechanism for the capture of secreted product which permits products secreted by eukaryotic and prokaryotic cells or cell aggregates to be captured at the surface of the cell. The captured product permits the cell to be detected analyzed and if desired, sorted, according to the presence, absence or amount of the product present. The means of capture comprises a specific binding partner for the product which has been anchored to the cell surface by a means suitable for the cell to be sorted. As used herein, the term “cell” or “cells” include cell aggregates; cell aggregates are groups of cells that produce a designated secreted product and are known in the art, and include, for example, the islets of Langerhans. The specific binding partner may be coupled to the anchoring means through a linking agent, and may also include a linker which multiplies the number of specific binding partners available and thus the potential for capture of product, such as branched polymers, including, for example, modified dextran molecules, polyethylene glycol, polypropylene glycol, polyvinyl alcohol, and polyvinylpyrrolidone.

For cells without cell walls, such as mammalian or other animal cells or cell protoplasts, suitable anchors to the cell surface include lipophilic molecules such as fatty acids. Alternatively, antibodies or other specific binding agents to cell surface markers such as the MHC antigens or glycoproteins, could also be used. For cells which have cell walls, such as plant cells, fungi, yeast or bacteria, suitable anchoring devices include binding agents to cell wall components, including, for example, antibodies.

Specific binding partners for the cell product to be captured include any moiety for which there is a relatively high affinity and specificity between product and its binding partner, and in which the dissociation of the product:partner complex is relatively slow so that the product:partner complex is detected during the cell separation technique. Specific binding partners may include, for example, substrates or substrate analogs to which a product will bind, peptides, polysaccharides, steroids, biotin, Digitoxin, Digitonin and other molecules able to bind the secreted product, and in a preferred embodiment will include antibodies. As used herein, the term “antibody” is intended to include polyclonal and monoclonal antibodies, chimeric antibodies, haptens and antibody fragments, and molecules which are antibody equivalents in that they specifically bind to an epitope on the product antigen.

In the practice of the invention the specific binding partner that entraps the product can be attached to a cell membrane (or cell wall) by a variety of methods. Suitable methods include, for example, direct chemical coupling to amino groups of the protein components, coupling to thiols (formed after reduction of disulfide bridges) of the protein components, indirect coupling through antibodies (including pairs of antibodies) or lectins, anchoring in the lipid bilayer by means of a hydrophobic anchor, and binding to the negatively charged cell surface by polycations. In other embodiments of the invention, the specific binding partner on the cell surface is introduced using two or more steps, e.g., by labeling the cells with at least one anchoring partner moiety which allows the coupling of the specific binding partner (with the capture moiety) to that moiety or another moiety(s) that link the moiety bound to the cell to the partner containing the capture moiety.

Methods for direct chemical coupling of antibodies to the cell surface are known in the art, and include, for example, coupling using glutaraldehyde or maleimide activated antibodies. Methods for chemical coupling using multiple step procedures include, for example, biotinylation, coupling of Trinitrophenol (TNP) or Digoxigenin using for example succinimide esters of these compounds. Biotinylation may be accomplished by, for example, the use of D-biotinyl-N-hydroxysuccinimide. Succinimide groups react effectively with amino groups at pH values above 7, and preferentially between about pH 8.0 and about pH 8.5. Biotinylation may also be accomplished by, for example, treating the cells with dithiothreitol followed by the addition of biotin maleimide.

Coupling to the cells may also be accomplished using antibodies against cell surface antigens. Antibodies directed to surface antigens generally require in the range of 0.1 to 1 μg of antibody per 10⁷ cells.

Cells carrying large amounts of N-acetylneuraminic acid on their surface as a constituent of their lipopolysaccharides bear a negative charge at physiological pH values. Coupling of capture moieties may be via charge interactions. For example, moieties bearing polycations bind to negatively charged cells. polycations are known in the art and include, for example, polylysine and chitosan. Chitosan is a polymer consisting of D-glucosamine groups linked together by β-(1-4) glucoside bonds.

Another method of coupling binding partners (also referred to herein as “capture moieties”) to the cells is via coupling to the cell surface polysaccharides. Substances which bind to polysaccharides are known in the art, and include, for example, lectins, including concanavalin A, solanum tuberosum, aleuria aurantia, datura stramonium, galanthus nivalis, helix pomatia, lens culinaris and other known lectins supplied by, a number of companies, including for example, Sigma Chemical Company and Aldrich Chemical Company.

In some embodiments of the invention, the product binding partner is coupled to the cell by hydrophobic anchoring to the cell membrane. Suitable hydrophobic groups that will interact with the lipid bilayer of the membrane are known in the art, and include, for example, fatty acids and non-ionic detergents (including, e.g., Tween-80). A drawback to attachment of the capture moiety to the cell via the insertion of a hydrophobic anchor is that the rate of integration of the hydrophobic moiety into the cell is low. Thus, high concentrations of the moiety with the hydrophobic anchor often are required. This latter situation is often uneconomical when the capture moiety is a relatively limited or expensive substance, for example, an antibody.

The low yield of hydrophobic molecules that embed themselves in the membrane is relevant only when these molecules are available in relatively limited quantities. This problem may be overcome by using a bridging system that includes an anchoring partner and a partner that contains the capture moiety, wherein the one of the partners is of higher availability, and wherein the two parts of the bridging system have a high degree of specificity and affinity for each other. For example, in one embodiment avidin or streptavidin is attached to the cell surface via a hydrophobic anchor, while the partner with the product capture moiety are biotinylated anti-product antibodies. In another embodiment, the cell surface is labeled with Digoxigenin followed by conjugates of anti-Digoxigenin antibody fragments and anti-product antibodies. This approach can be used with other pairs of molecules able to form a link, including, for example, hapten with antihapten antibodies, NTA with polyhistidine residues, or lectins with polysaccharides. A preferred embodiment is one which allows “amplification” of the system by increasing the number of capture moieties per anchoring moiety.

In one illustrative embodiment, a branched dextran is bound to palmitic acid, thus providing a multiplicity of available binding sites. The dextran is in turn coupled to biotin and treated with avidin-conjugated antibody specific for the product secreted by the cell.

It is of course contemplated within the embodiments of the invention that bridging systems may be used between the anchoring moiety and the capture moiety when the anchoring moiety is coupled in any fashion to the cell surface. Thus, for example, an avidin (or streptavidin) biotin bridge may link an antibody anchor moiety with an antibody capture moiety. Bivalent antibody systems may also act as bridging systems.

In order to analyze and, if desired, to select cells that have the capability of secreting the product of interest, cells modified as above to contain the capture partner moiety are incubated under conditions that allow the production and secretion of the product in a sufficiency to allow binding to and detection of the cells that contain the captured product. These conditions are known to those of skill in the art and include, inter alia, appropriate temperature, pH, and concentrations of salts, growth factors and substrates in the incubation medium, as well as the appropriate concentrations of gas in the gaseous phase. When it is desirable to distinguish between high and low producer cells, the time of incubation is such that product secretion by the cells is still in a linear phase. Additionally, the cells can be stimulated using extra factors or extra stimulating cells that cause the potential producer cells to be in a special desired functional state. Generally the incubation time is between 5 minutes and ten hours, and more usually is between 1 and 5 hours.

The incubation conditions are also such that product secreted by a producer cell is essentially not captured by another cell, so distinguishing non-producing cells from product producing cells, or high producers from low producers is possible. This may be accomplished by, for example, including in the incubation medium a substance that slows the diffusion of the secreted product from the producer cell. Substances which inhibit product diffusion in liquid media and that are non-toxic to cells are known in the art, and include, for example, a variety of substances that partially or completely gel, including, for example, low melting agarose and gelatin. Preferably, the gels are soluble after the incubation to allow the isolation of the cells or groups of cells from the media by cell sorting techniques. Thus, for example, the gels may be linked by disulfide bonds that can be dissociated by sulfhydryl reducing agents such as β-mercaptoethanol or dithiotreitol, or the gels may be contain ion cross-linkings, including for example, calcium ions, that are solubilized by the addition of a chelating agent such as EDTA.

In a preferred embodiment, during the secretion phase the cells are incubated in a gelatinous medium, and preferentially the size limitation of penetration into the gel prevents the product from substantially entering the gel.

At the end of the secretion phase the cells are usually chilled to prevent further secretion, and the gel matrix (if any) is solubilized. This order may, of course, be reversed. The cells containing the trapped product are then labeled. Labelling may be accomplished by any method known to those of skill in the art. For example, anti-product antibodies may be used to directly or indirectly label the cells containing the product. The labels used are those which are suitable for use in systems in which cells are to be analyzed or sorted based upon the attachment of the label to the product.

In other embodiments, unlabelled product capture partners on the cell surface may be detected. This allows, for example, the isolation of cells that secrete high amounts by employing a negative separation method, i.e., detection of cells not highly saturated with product. The cells can be stained with other labeling substances recognizing, e.g., cell type, cellular parameters such as DNA content, cell status, or number of capture moieties. The enumeration of actual capture moieties can be important to compensate for varying amounts of these molecules due to, for example, different conjugation potentials of the cells. It may be especially important for the isolation of rare cells to exclude cells with decreased or increased capability for binding the product capture system, including the anchoring and binding moieties.

Analysis of the cell population and cell sorting based upon the presence of the label may be accomplished by a number of techniques known in the art. Cells can be analyzed or sorted by, for example, Flow Cytometry or Fluorescence activated cell sorting (FACS). These techniques allow the analysis and sorting according to one or more parameters of the cells. Usually one or multiple secretion parameters can be analyzed simultaneously in combination with other measurable parameters of the cell, for e.g., cell type, cell surface antigens, DNA content, etc. The data can be analyzed and cells can be sorted using any formula or combination of the measured parameters. Cell sorting and cell analysis methods are known in the art and are described in, for example, THE HANDBOOK OF EXPERIMENTAL IMMUNOLOGY, Volumes 1 to 4, (D. N. Weir, editor) and FLOW CYTOMETRY AND CELL SORTING (A. Radbruch, editor, Springer Verlag, 1992). Cells can also be analyzed using microscopy techniques including, for example, laser scanning microscopy, fluorescence microscopy; techniques such as these may also be used in combination with image analysis systems. Other methods for cell sorting include, for example, panning and separation using affinity techniques, including those techniques using solid supports such as plates, beads and columns.

Some methods for cell sorting utilize magnetic separations, and some of these methods utilize magnetic beads. Different magnetic beads are available from a number of sources, including for example, Dynal (Norway), Advanced Magnetics (Cambridge, Mass., U.S.A.), Immuncon (Philadephia, U.S.A.), Immunocon (Marseille, France), and Miltenyi Biotec GmbH (Germany).

Preferred magnetic labeling methods include colloidal superparamagnetic particles in a size range of 5 to 200 nm, preferably in a size of 10 to 100 nm. These magnetic particles allow a quantitative magnetic labelling of cells, thus the amount of coupled magnetic label is proportional to the amount of bound product, and the magnetic separation methods are sensitive to different amounts of product secretion. Colloidal particles with various specificities are known in the art, and are available, for example, through Miltenyi Biotec GmbH. The use of immunospecific fluorescent or magnetic liposomes may also be used for quantitative labeling of bound product. In these cases, the liposomes contain magnetic material and/or fluorescent dyes conjugated with antibody on their surfaces, and magnetic separation is used to allow optimal separation between nonproducing, low producing, and high producing cells. The magnetic separation can be accomplished with high efficiency by combining a second force to the attractive magnetic force, causing a separation based upon the different strengths of the two opposed forces. Typical opposed forces are, for example, forces induced by magnetic fluids mixed in the separation medium in the magnetic separation chamber, gravity, and viscous forces induced by flow speed of medium relative to the cell. Any magnetic separation method preferably magnetic separation methods allowing quantitative separation will be used. It is also contemplated that different separation methods can be combined, for example, magnetic cell sorting can be combined with FACS, to increase the separation quality or to allow sorting by multiple paramters.

Preferred techniques include high gradient magnetic separation (HGMS), a procedure for selectively retaining magnetic materials in a chamber or column disposed in a magentic field. This technique can also be applied to non-magnetic targets labeled with magnetic particles. In one application of this technique the target material is labeled by attaching it to a magnetic particle. The attachment is generally through association of the target material with a specific binding partner which is conjugated to a coating on the particle which provides a functional group for the conjugation. The material of interest, thus coupled to a magnetic “label”, is suspended in a fluid which is then applied to the chamber. In the presence of a magnetic gradient supplied across the chamber, the magneticallly labeled target is retained in the chamber; if the chamber contains a matrix, it becomes associated with the matrix. Materials which do not have or have only a low amount of magnetic labels pass through the chamber. The retained materials can then be eluted by changing the strength of, or by eliminating, the magnetic field or by introducing a magnetic fluid. The selectivity for a desired target material is supplied by the specific binding-partner conjugated to the magnetic particle. The chamber across which the magnetic field is applied is often provided with a matrix of a material of suitable magnetic susceptibility to induce a high magnetic field gradient locally in the camber in volumes close to the surface of the matrix. This permits the retention of fairly weakly magnetized particles. Publications describing a variety of HGMS systems are known in the art, and include,for exmaple, U.S. Pat. NoS. 4,452,773, 4,230,685, PCT application WO85/04330, U.S. Pat. No. 4,770,183, and PCT/EP89/01602; systems are also described in U.S. Ser. No. 07/291,177 and in U.S. Ser. No. 07/291,176, which are commonly owned and hereby incorporated herein by reference.

As seen from above, processes embodied by the invention include the following steps:

a. Coupling an anchoring partner to the surface of the cells suspected of secreting a designated product;

b. coupling to the anchoring partner a capture partner which captures secreted product; and

c. incubating the cells with the coupled partners to allow synthesis and secretion of the designated product.

In addition, in other embodiments the processes include labeling the cells that contain the product captured by the capture moiety, if any. Other embodiments may also include analyzing the cell population to detect labeled cells, if any, and if desired, sorting the labeled cells, if any.

The processes of the invention may be used to analyze and/or separate a variety of types of cells. For example, it can be used to detect and select hybridoma cell lines that are high level secretors.

An exemplary process for the selection of this type of hybridoma cell is the following. The cells are modified to contain a digoxygenin anchor moiety by coupling digoxygenin to the cell via a lipophilic anchor or by chemical coupling. A product capture moiety is linked to the cells via a rat anti-kappa or rat anti-lambda monoclonal antibody conjugated to anti-digoxygenin antibody or antibody fragments. The cells with the linked capture moiety are incubated to allow secretion of the monoclonal antibodies. Cells trapping the secreted product antibodies are labeled by incubation with rat anti-mouse IgG1 or IgG2a+b monoclonal antibody. An anti-class antibody that does not recognize the surface bound form of the product is advantageous when the expression product is naturally associated with the cell surface.

Selection of the high secretor cells is carried out in multiple rounds. Each separation process involves the use of a cell separation process, i.e., a quantitative magnetic separation system that distinguishes different levels of bound product, or a fluorescence actived cell sorter (FACS. The cells having the highest labeling (eventually normalized on a cell to cell basis using further parameters) are sorted and expanded in culture again. Magnetic and FACS separation can be combined. FACS sorting is preferentially performed by additionally staining the cells for amount of capture antibody using a different fluorochrome than that with which the cells are originally stained, then selecting for cells with a high ratio of amount of product to amount of antibody. Multliple rounds of separation using high cell numbers of 10⁷ to 19¹⁰ cells allows isolation of rare genetic variants showing extraordinarily high levels of production and genetic stability. In order to avoid the selection of cells producing aberrant forms of product, different selection antibodies may be used during the different rounds of separation.

Using a similar approach hybridomas with defined specificity may also be detected and selected. By employing a selection process on large cell numbers, rare genetic variants with higher affinity or specificity can be obtained. Class switch variants can be isolated using different anti-class antibodies. Generally, this approach can be used for the isolation of almost any kind of variant of the antibodies with the desired specificity.

The identification of and isolation of genes coding for a specific substance, and the isolation of cells producing a specific protein, including specific fusion proteins, cytokines, growth hormones, viral proteins, bacterial proteins, etc., can also be accomplished using the processes of the invention. For example, if it is desirable to select for a cell producing a specific protein, the cells can be genetically modified by the introduction of an expression vector that encodes the protein of interest. The cells are modified by the introduction of a product capture system, including a capture moiety specific to the product and anchor moiety, and the cells are grown under conditions that allow product secretion. The cells containing the captured product are labeled, and subjected to one or more rounds of separation based upon the presence of label.

The process of the invention may also be used to simultaneously analyze qualitative and quantitative secretion patterns in complex cell mixtures such as, for example, mixtures containing white blood cells, or bone marrow cells, or suspended tumor or tissue cells. In this case, the cells in the mixture would be labeled with cell specific markers, and would also be labeled with capture moieities for the products to be detected. After the secretion phase, the cells would be subjected to multiparameter analysis as used in flow cytometry and/or image analysis, and the results analyzed with multi-dimensional statistical methods known in the art, and used in the analysis of flow cytometry and image analysis data. If the analysis is to determine cells specifically reactive with a factor or cell (for example hormones, antibodies against a cell receptor, or cancer cells) the cells to be analyzed can be exposed to these factors or cells during the incubation period prior to analysis by flow cytometry or image analysis. Methods such as these are potentially of value for various diagnostic applications in medicine, for example, for measuring levels and types of interleukin production in various cell populations, and for measuring growth factor release in tumor cell populations.

It is contemplated that the reagents used in the detection of secretor cells of desired products may be packaged in the form of kits for convenience. The kits would contain, for example, one or more materials for use in preparing gelatinous cell culture medium, the medium to be used for cell incubation for the production of the desired secreted product; a product capture system comprised of anchor and capture moieties; a label for detecting the captured product; and instructions for use of the reagents. All the reagents would be packaged in appropriate containers.

The Examples described below are provided only for illustrative purposes, and not to limit the scope of the present invention. In light of the present disclosure, numerous embodiments within the scope of the claims will be apparent to those of ordinary skill in the art.

### EXAMPLES

### Example 1

The purpose of this example was to separate living cells that secrete a given product from a mixture of externally identical cells. The B.1.8. hybridoma cell line and the X63. Ag 8 6.5.3. myeloma cell line were used as the test system. About 60% of B.1.8. cells secrete IgM; the myeloma line secretes no protein. The secreting cells were to be separated from B.1.8. and from mixtures of B.1.8. with Ag 8 6.5.3. cells. To achieve this, a procedure was developed to trap a product secreted by a cell on the surface, hold it there, and thus label the cell in-question. The entrapment antibodies were attached in two steps: 1) biotinylation of the cells; and 2) attachment of the entrapment antibody through an avidin-biotin coupling reaction. The labeled cells were then separated from cell mixtures.

### Biotinylation of the Cell Surface with Biotinylpalmitoyldextran

The objective was the synthesis of a large macromolecule with biotin groups and fatty acid groups that was to embed itself in the cell membrane.

Synthesis of a Hydrophobic Biotin

A dextran with a molecular weight of 3×10⁶ g/mole was used as the carrier molecule. In order to be able to couple both biotin groups and the fatty acid group to the polysaccharide, reactive primary amino groups first were introduced into the dextran.

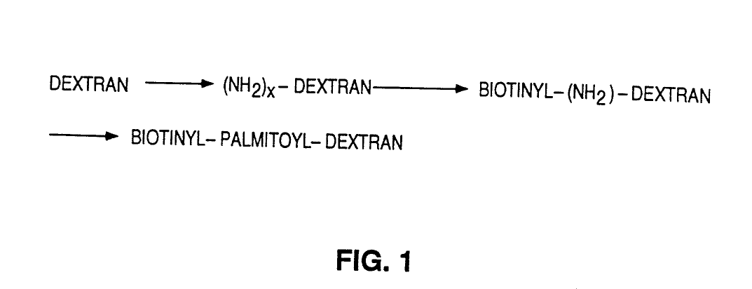

Biotinyl groups and a palmitoyl group were then to be introduced to the amino groups of proteins by somewhat modified methods such as those used for coupling biotin and fatty acid esters. FIG. 1 shows the scheme for the introduction of biotinyl and palmitoyl groups onto Dextran.

Synthesis of an Aminodextran

Amino groups were introduced into Dextran molecules by activation with carbodiimidazole and reaction with diaminohexan using standard methods. Aminodextran was obtained from Sigma Corp. and from Molecular Probes (Oregon). An aminodextran with 165±20 amino groups per molecule of 3×10⁶ g/mole was obtained. Polymerization of dextran occurs as a side reaction. The yield of unpolymerized product amounted to 94% of the starting dextran.

A method described by Dubois was used to determine dextran concentrations. 5 μl of an 80% solution of phenol in water was placed in a test tube with 100 μl of the dextran solution to be determined. 1 ml of concentrated sulfuric acid was pipetted quickly into this mixture. After 10 minutes, the formulation was placed in a water bath at 30° C. for 10 minutes longer. The dextran concentration was found by measuring the extinction at 480 nm.

Synthesis of Biotinylaminodextran

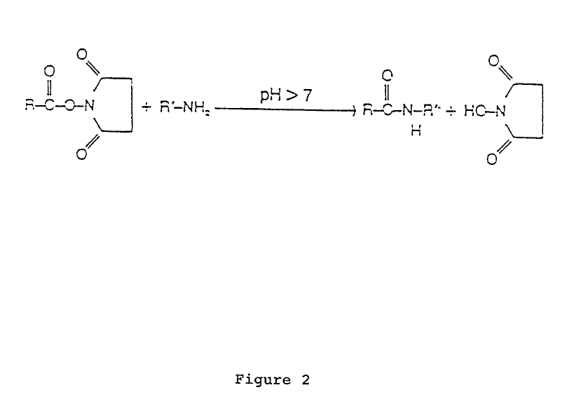

The introduction of biotinyl groups onto the dextran was accomplished using D-biotinyl-N-hydroxysuccinimide as the biotinylation reagent. Succinimide groups react effectively with amino groups at pH values above 7. FIG. 2 is a scheme for the reaction of N-hydroxysuccinimide esters with primary amino groups in basic form. The corresponding N-hydroxysuccinimide esters were used for introducing both the biotinyl groups and the palmitoyl groups in the dextran. In this Figure, R′ stands for dextran, and R stands for either a biotinyl group or for a palmitoyl group.

Synthesis of Biotinylpalmitoyldextran

Palmitic acid groups were coupled to the biotinylated dextran. The reaction was carried out by a slightly modified procedure for coupling palmitoyl groups to antibodies (Huang et al., J. Biol. Chem. 255:8015-8018 (1980). The coupling occurs similarly to the preceding biotinylation by nucleophilic attack of the amino groups of the dextran on the N-hydroxysuccinimide ester of palmitic acid.

### Biotinylation of Cells with Biotinylpalmitoyldextran

The ability of the lipopolysaccharide, biotinylpalmitoyldextran, to bind to cells and thereby biotinylate the cell surface was tested on human lymphocytes and compared with the binding of biotinylaminodextrans lacking palmitoyl groups.

The cells were centrifuged out at 20° C. and incubated for 10 minutes at 37° C. with 1 mg/ml of either biotinyldextran or biotinylpalmitoyldextran in PBS (100 μl for 10⁷ cells). 1 ml of PBS 1% BSA was then added, and after 3 minutes the cells were washed on ice in 14 ml of PBS. The treated cells were taken up in PBS 0.03% azide.

Biotinylation of the cells by biotinyldextran or biotinylpalmitoyldextran was monitored by staining of the cells with streptavidin-FITC. More specifically, the treated cells were washed and taken up in 100 μl of PBS/10⁷ cells. 1 μl of 100 μg/ml fluorescein-conjugated streptavidin in PBS was added and the mixtures were incubated for 5 minutes on ice. The cells were then washed, taken up in 1 ml of PBS 1% BSA per 10⁷ cells, and the intensity of fluorescence was measured in the FACScan as a measure of biotinylation.

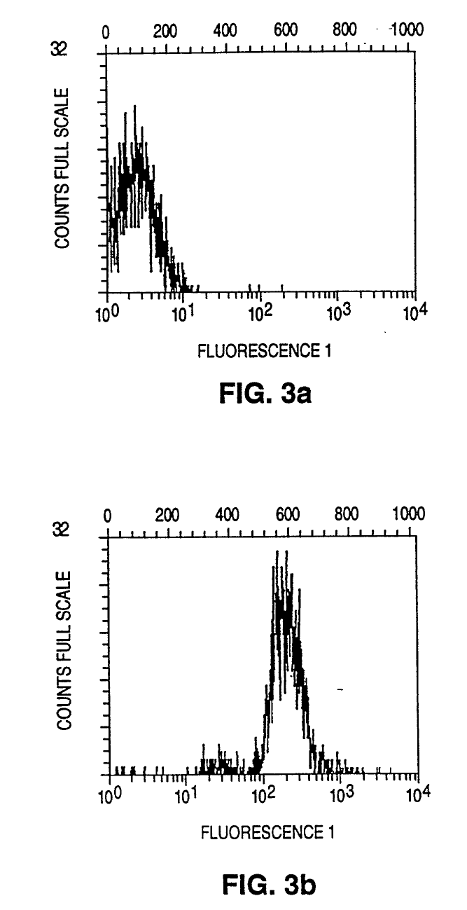

The results of the FACScans of binding of streptavidin to cells treated with biotinyldextran and biotinylpalmitoyldextran are shown in FIG. 3a and FIG. 3b, respectively. As seen from the results, cells incubated with biotinyldextran did not bind streptavidin. In contrast, cells incubated with biotinylpalmitoyldextran bound large amounts of the streptavidin-FITC.

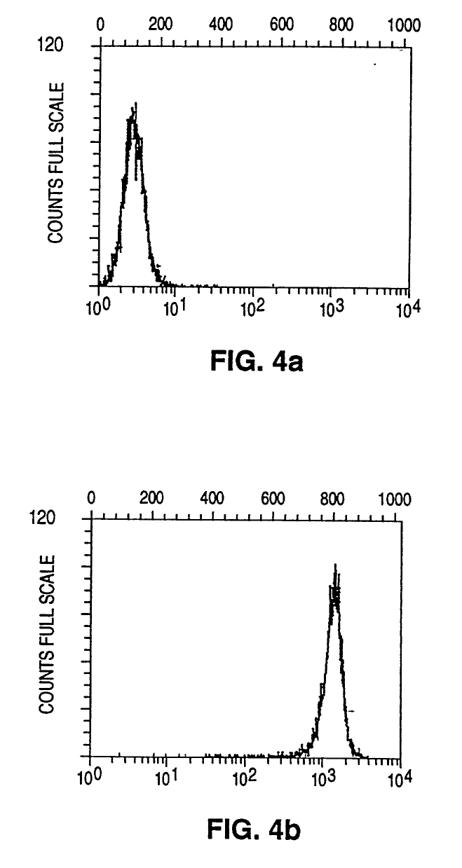

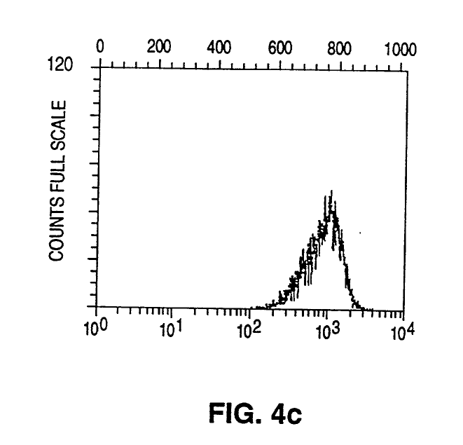

A comparison was made between the amount of streptavidin-FITC bound by cells labeled with biotinyl-antibodies directed towards β-2 microglobulin and the biotinylpalmitoyldextran labeled cells. The antibody used was αβ₂m, an antibody that binds to β₂ microglobulin (β₂m). FIG. 4 shows traces of the FACScan results: a) unbiotinylated cells treated with streptavidin-FITC (negative control); b) cells treated with biotinylpalmitoyldextran and then with streptavidin-FITC; c) cells incubated with biotinyl-anti-αβ₂m and treated with streptavidin-FITC. The results in FIG. 4 indicate that cells labeled with biotinylpalmitoyldextran are able to bind more streptavidin to the cell surface than an αβ₂m-biotin conjugate.

While antibody staining of the cell reaches saturation, staining by biotinylpalmitoyldextran increases linearly with the concentration of the staining reagent. However, the staining is limited by injury to the cells when the concentrations of reagent are too high. When biotinylation of the cells was with about 1 mg/ml of biotinylpalmitoyldextran for 10 minutes at 37° C., no change of the cell surface was observed under the microscope; the light-scattering properties of the cell surface, which were measured in the FACScan with forward and lateral scattered light, were unchanged compared to untreated cells. The treated cells maintained viability and could be cultured again.

### Coupling of Entrapment Antibodies to Biotinylated Cells

Entrapment antibodies were coupled to cells biotinylated with biotinylpalmitoyldextran via an avidin-biotin bridge. In order to accomplish this, the entrapment antibodies were conjugated with avidin, and the conjugates reacted with the biotinylated cells.

Two antibodies, rat anti-mouse IgM (R33.24.12) and mouse kappa against mouse lambda (LS136) against various epitopes on mouse IgM (lambda) were coupled to avidin.

Avidin is a basic protein with several reactive amino groups. Succinimydyl 4-(N-maleimidomethyl) cyclohexane-1-carboxylate (SMCC) was used to couple avidin to the entrapment antibodies. SMCC is a bivalent linker molecule whose maleimide group reacts selectively with thiols and whose succinimdydyl group reacts selectively with primary amines. The entrapment antibody was reduced with dithiothreitol (DTT). DTT is a mild reducing agent that under suitable conditions reduces 1-4 disulfide bridges of an IgG molecule to thiols without destroying the antigen-binding site. A reactive maleimide group was introduced on the amino groups of avidin with SMCC. The maleimide group of avidin was reacted with the SH groups of the reduced antibody. Avidin and entrapment antibodies were joined in this way through a cyclohexane bridge.

More specifically, 1.5 μl of a 1 molar solution of DTT was added to 1 milligram of antibody of the IgG class in 200 μl of PBS containing 5 mMEDTA. After reaction for 1 hour at room temperature, the reduced antibody was placed on a Sephadex PD10 column and eluted in 1 ml of PBS/EDTA. The number of thiol groups introduced per antibody molecule was determined. The desirable range is about 2-6 thiol groups per antibody molecule.

Concomitantly, one milligram of avidin was dissolved in 100 μl of carbonate buffer pH=9.4 and 125 μg of SMCC in 7.5 μl of DMSO were added. After 1 hour at room temperature, the protein was purified on a Sephadex PD10 column and taken up in 500 μl of PBS/EDTA.

One milligram of the reduced antibody in 1 ml of PBS/EDTA was combined with 400 μg of the activated avidin in 200 μg of PBS/EDTA and allowed to stand overnight at 4° C. The reaction was stopped by adding 5← μl of one molar N-ethylmalemide.

Coupling of Avidin-labeled Entrapment Antibodies to Biotinylated Cells

A mixture of myeloma and hybridoma cells was used. B.1.8. hybridoma cells that secrete IgM and nonproducing X63. Ag 8 6.53. myeloma cells were grown at 37° C. in an atmosphere saturated with water vapor. The culture medium contained RPMI and 5% fetal calf serum, 100 IU/ml of penicillin, and 0.1 mg/ml of streptomycin.

The cell mixture was biotinylated with biotinylpalmitoyldextran using the conditions described above.

In order to couple the antibody-avidin conjugates to the biotinylated cells, the biotinylated cells, after washing in PBS/1% bovine serum albumin (BSA), were incubated with an avidin-entrapment antibody conjugate. 1 μl of a solution of 1 mg/ml of entrapment antibody-avidin conjugate in PBS was added to 10⁷ biotinylated cells in 100 μl of PBS 0.03% azide. After 10 minutes on ice, the biotin groups were saturated with entrapment antibody, and the cells were loaded with entrapment antibodies.

In order to detect the presence of the avidin-antibody complexes on the cell surface, a fluorescent anti-antibody was used, and the fluorescent labeling detected by FACScan. The staining of cells corresponded approximately to the staining of biotinylated cells with fluorescent streptavidin, performed in the same study. A uniform staining of the cell population was observed; all of the cells carried about the same amounts of entrapment antibodies on their surfaces.

Testing the Functionality of Entrapment Antibodies on the Cell Surface

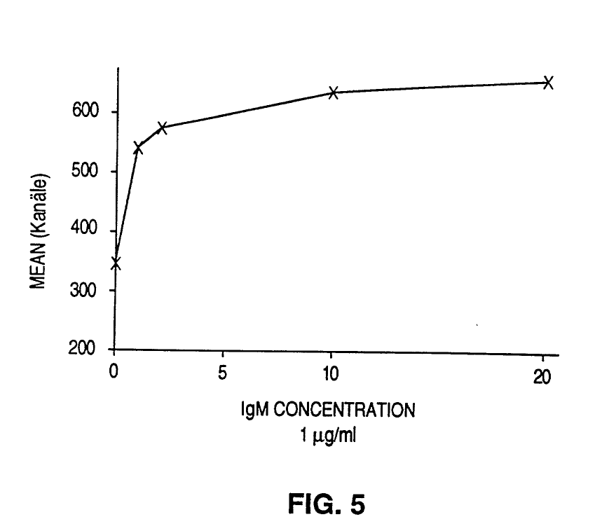

About 10⁷ cells provided with entrapment antibodies were incubated on ice (so that they secreted no protein) in 100 μl PBS/1% BSA, with various concentrations of mouse IgM, which is trapped by the entrapment antibodies. After 5 minutes of incubation the cells were washed and the trapped IgM was detected on the cell surface using R-PE conjugate as the antibody label. Detection was in the FACScan. FIG. 5 shows the titration curve. In the figure, the fluorescence of the cells (mean) is plotted against the IgM concentration with which the cells were incubated. These results show that the entrapment antibody on the cell surface still has intact binding sites. The sensitivity of the entrapment antibodies to low IgM concentrations in the medium is also recognizable. The titration curve illustrated was obtained using R33.24.12 as entrapment antibody. The entrapment antibody LS136 was used for the entrapment experiments shown later. The latter showed somewhat higher sensitivity to low IgM concentrations in the medium.

Entrapment of Secreted IgM

A mixture of biotinylated B.1.8. and X63 cells was conjugated with entrapment antibodies and was kept under a 7.5% CO₂ atmosphere at 37° C. for various lengths of time in medium. The IgM trapped on its surface was then detected by an antibody stain.

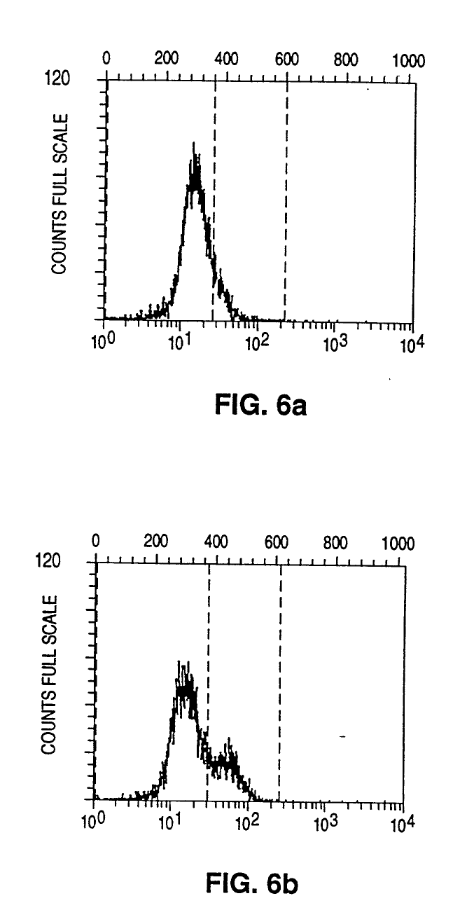

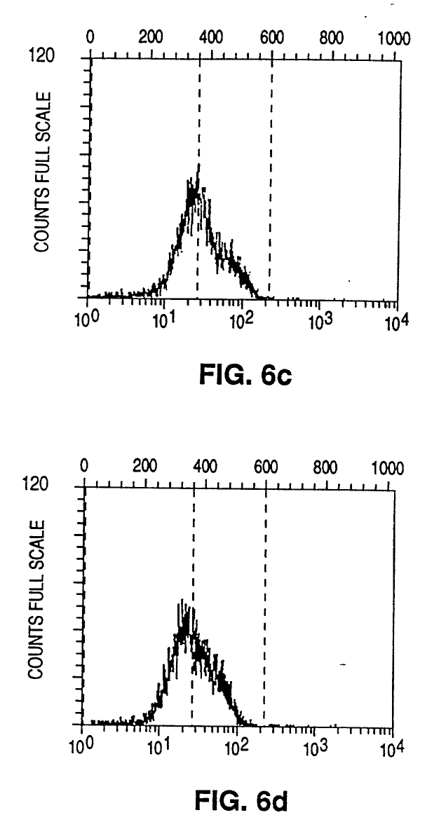

FIG. 6 shows the resulting stainings as FACScan illustrations: duration of entrapment test (6a) 10 min; (6b) 30 min; (6c)1 h; and (6d) 2 h. Two populations can be differentiated after 30 min, which have captured different amounts of IgM. The difference between the two populations disappears after lengthy incubation because of IgM given off to the medium by the secreting cells, which is taken up by the entrapment antibodies on the nonsecreting cells.

Entrapment of Secreted IgM Using a Diffusion Inhibitor

It can be seen from the illustration above that the less strongly stained cell population also takes up IgM rapidly on its surface. This background staining comes from secreted IgM in the culture medium that has not been trapped by the entrapment antibodies on the secreting cells. If the entrapment experiment is carried out in a more viscous medium, this background staining can be reduced. Culture medium with 2.5% methylcellulose was used; this medium shows a gellike consistency.

The cells loaded with entrapment antibodies were mixed in culture medium with 2.5% methylcellulose or 1% methylcellulose. It was unnecessary and superfluous to wash the cells; entrapment antibody-avidin conjugate not bound to the cells does not interfere. 2 ml of medium was used for 10⁷ cells. To bring the methylcellulose properly into solution, it was admixed with the culture medium one day previously. The medium was preheated to 37° C., the cells were added and were incubated for 25 to 45 minutes at 37° C. with 7.5% CO₂. Under these conditions, the hybridoma cells secreted their product. After the incubation time, the high-viscosity medium was diluted with 45 ml of cold PBS 1% BSA. The cells were centrifuged out at 4° C. and taken up in 100 to 500 μl of PBS 1% BSA. Remainders of methylcellulose gave the cell suspension an elevated viscosity; neither the cells nor the subsequent staining steps were harmed by this.

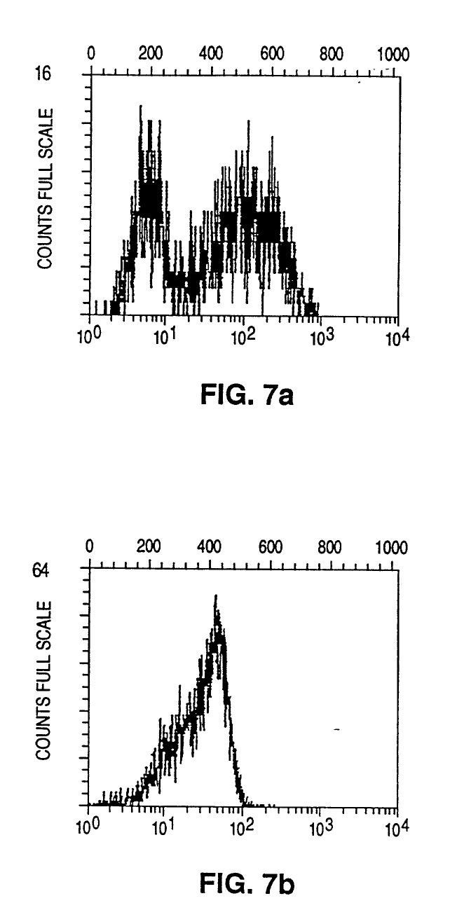

FIG. 7 shows the effect of the concentration of methylcellulose in the medium on entrapment. The cells in this experiment produced IgM for 35 minutes in 2.5% methylcellulose, and were then washed and stained with R33.24.12. R-PE. FIGS. 7a and 7b show the entrapment of antibodies by cells incubated in 2.5% and 1% methylcellulose medium, respectively. The results indicate that the secreting and non-secreting cells were successfully distinguished based on entrapment in the 2.5% methylcellulose medium.

Double Staining

To show that the cells carrying IgM on their surface after the entrapment experiment described above actually are cells secreting IgM, the cells were stained red on the surface after the entrapment experiment with R33.24.12. R-PE (visible in the FACScan as Fluorescence 2.), fixed, and stained green in the cytoplasm with R.33.24.12.-FITC (visible in the FACScan as Fluorescence 1.). The cells stained twice in this way were examined under the microscope and in the FACScan.

The fact that the cells carrying IgM on their surface after the entrapment experiment are secreting cells was illustrated by this double staining as a two-dimensional representation of Fluorescence 1 and 2 in the FACScan. B.1.8. cells after the entrapment experiment were stained red on their surface relative to the trapped IgM (visible in the FACScan as Fluorescence 2), and were then fixed and stained green in the cytoplasm relative to IgM (visible in the FACScan as Fluorescence 1). All of the surface-stained cells are also stained in the cytoplasm.

The results indicated that all of the cells not producing IgM also belonged to the cell population that were not surface-stained. The cytoplasm-positive cells were divided into two fractions; on the one hand, a fraction of cells stained both on the surface and in the cytoplasm. These were apparently secreting cells. On the other hand, a cell fraction was stained in the cytoplasm but not on the cell surface. Since this population could not be separated by a Ficoll gradient (carried out just before fixation), they were not dead cells. Some of the cells in this population were also not stained as intensely in the cytoplasm as the secreting cells. The broader dispersion of this fraction compared to the two other cell populations was also striking. These cells produced IgM but apparently lost the ability to secrete this protein. The double-stained cells were observed under the microscope as a control. This examination showed conformity with the outcome of the FACScan representation.

Cell Separation of the MACS

After the entrapment of secreted IgM with a 1:1 mixture of about 10⁷ B.1.8. and X63 cells, separations were carried out with the magnetic cell sorting system (MACS), using magnetic particles that bind to the trapped IgM on the cell surface. The MACS system and magnetic particles were from Miltenyi Biotec GmbH (Germany).

A mixture of IgM-secreting and nonsecreting cells was provided with the matrix for trapping secreted IgM developed in this work and was kept at 37° C. in an atmosphere of 7.5% CO₂ for 25 minutes in 5 ml of culture medium with 2.5% methylcellulose. The cells were washed in 45 ml of PBS 1% BSA. The pellet was treated with a remainder of methylcellulose of gel-like consistency. It was taken up in 500 μl of PBS 1% BSA and 5 μl of rat antimouse IgM magnetic beads (Miltenyi Biotec GmbH) were added. After 5 minutes on ice, 10 μg/ml of R-PE-coupled R33.24.12 antibody was added and the mixture was kept on ice for 5 min longer.

About 10⁷ cells pretreated in this way were placed on a type A1 separating column in the MACS (Miltenyi Biotec GmbH) and the negative fraction was eluted with 10 ml of PBS 1% BSA at 5° C. After removing the column from the magnetic field, the positive cell fraction was eluted in 10 ml of PBS 1% BSA.

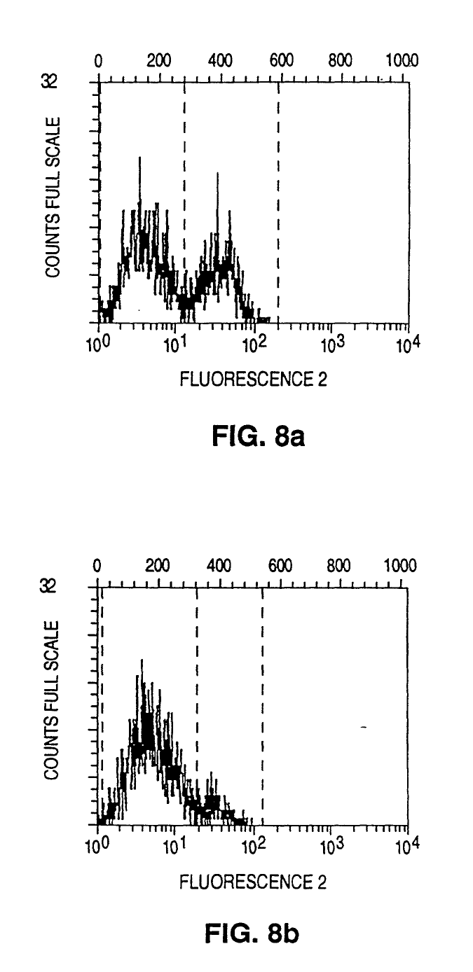

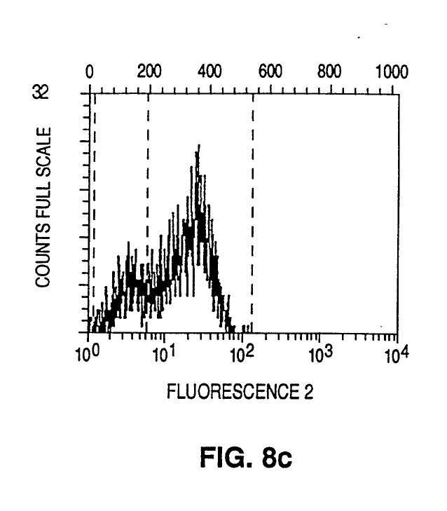

Cells surface-stained red relative to IgM are shown in FIG. 8 before and after the separation. The cell suspension prior to separation contained 58.8% unstained and 41.2% stained cells. After the separation the negative fraction contained 89% unstained and 11% stained cells. The positive fraction contained 23% unstained and 77% stained cells. When the concentration of positive cells after separation is calculated with the formula: concentration factor−(% pos. cells in pos. fraction*% neg cells in original cell mixture)/(% pos. cells in the original fraction*% neg cells in pos. fraction), a concentration factor of 4.8 is obtained.

After the separation, the cells could not be stained with propidium iodide, a dye that selectively stains dead cells, and could again be cultured. The vitality of the separated cell fractions was checked under the microscope one week after the separation. The loss of cells during the separation process was not determined; no relevant losses of cells normally occur in separations in the MACS.

FIG. 8 is a FACScan representation of stained cells before and after separations. After trapping secreted IgM, the cells were stained relative to IgM on the surface and were separated in the MACS with magnetic particles relative to IgM. The cells are shown in the FIG. 8a before separation. FIG. 8b shows the negative fraction after the separation. FIG. 8c shows the positive fraction after the separation. If the cells to the right of the broken line are considered to be stained and those on the left of it to be unstained, the cell fractions contained the following amounts of stained and unstained cells:

| | % | % |
|---|---|---|
| | neg. | pos. |
| Cells before separation | 58.8 | 41.2 |
| Negative fraction after | 89 | 11 |
| separation | | |
| Positive fraction after | 23 | 77 |
| separation | | |

The studies described above included the following general techniques.

Antibody Staining of Cells

The cells were taken up in PBS 1% BSA and pelleted by centrifugation. The supernatant was removed by suction, and the pellet resuspended in the antibody staining solution. 100 μl of staining solution containing 10-100 μg/ml of antibody in PBS 1% BSA 0.1% NaN₃ was used per 10⁷ cells. The coupling reaction was incubated 5 minutes on ice. The cells were then washed.

Ficoll Gradient Centrifugation

Ficoll gradient centrifugation was used to remove dead cells. The cell suspensions were carefully underlayered with 5 ml of Ficoll (Pharmacia LKB, Uppsala, Sweden) and were then centrifuged at 2500 rpm at room temperature. Living cells remained resting on the Ficoll cushion and were removed by suction.

Cytoplasm Staining

0.5% saponin and 10 μg/ml of staining antibody were added to the fixed cells in PBS 1% BSA. Saponin produces reversible channels about 10 nm in diameter in the cell membrane, so that the antibodies can penetrate into the cells. After a reaction time of 1 hour the cells were taken up in PBS 1% BSA 0.5% saponin (1 ml/10⁶ cells). After 30 minutes, the cells were washed and taken up in saponin-free PBS 1% BSA.

Antibody Used

R33.24.12., a monoclonal rat anti-mouse antibody, coupled both to R-PE and to fluorescein, was obtained from stocks of the Immunobiological Department of the Genetic Institute of Cologne. The optimal staining concentrations were titrated. The R-PE conjugate of this antibody was used to stain the trapped IgM on the surface of secreting cells, and the fluorescein conjugate was used for cytoplasm staining. LS136, a mouse IgG kappa against mouse lambda is used as the entrapment antibody (the IgM to be trapped is of the lambda allotype). LS136 likewise originates from the internal production of the Immunobiological Department.

### Example 2

This example demonstrates the effect of carrying out the secretion phase in a gelatinous medium as compared to a high viscosity medium on the capture (entrapment) of secreted product by cells containing a biotin anchor moiety linked via avidin to the capture antibody moiety.

Chemical Biotinylation of Cells Using NHS-LC-Biotin

A mixture of B.1.8. and X63 cells was chemically biotinylated by the following procedure. Cell suspensions containing 10⁷ to 10⁸ cells were centrifuged, the supernatant removed, and the pellet resuspended in a solution of 200 μl phosphate buffered saline (PBS) pH 8.5, containing 0.1 to 1 mg.ml NHS-LC-Biotin (Pierce, Rockford, Ill., U.S.A.). After incubation for 30 minutes at room temperature the cells were washed two times extensively with 50 ml PBS/BSA. Labeling with avidin conjugate was within 24 hours of the biotinylation.

Linkage of the Biotinylated Cells to Capture Antibodies with Avidin

The cells biotinylated by reaction with NHS-LC-Biotin were labeled with an avidin conjugate of LS136 (concentration of 30 μg/ml) for 30 minutes on ice and washed.

Secretion and Product Capture in Gelatinous Medium and in High Viscosity Medium

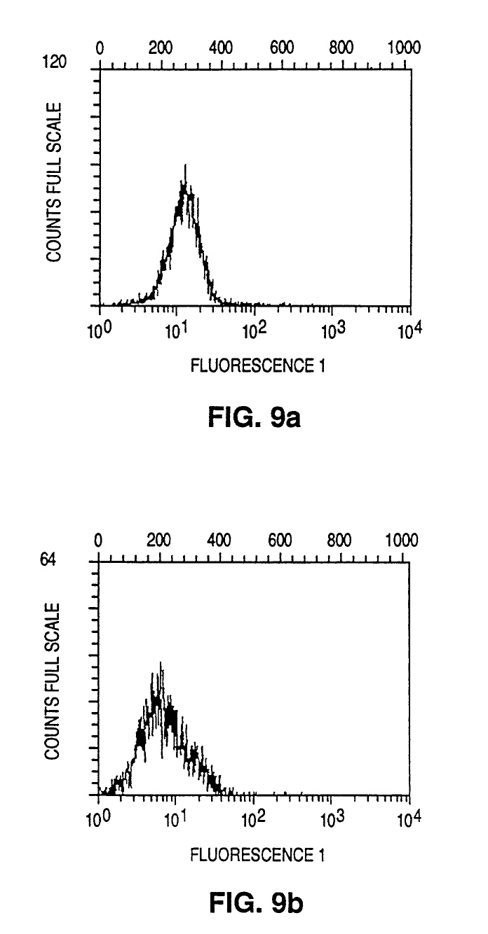

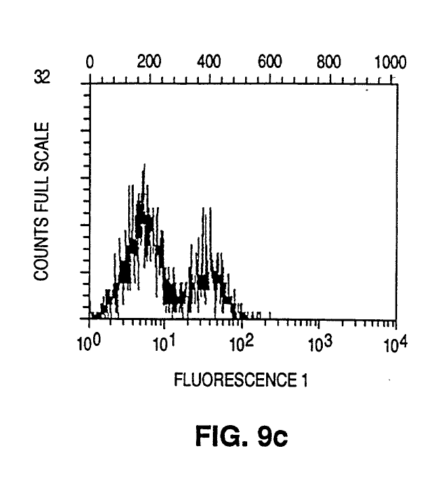

The biotinylated-avidin treated cells were incubated 1 hour at 37° C. under 7.5% CO₂ in three different media, washed and stained with a fluorescein conjugate of R 33.24.12 (10 μg/ml) for 10 minutes on ice, washed and analyzed using flow cytometry (FACScan) for determination of the amount of bound R 33.24.12. R 33.24.12 is a fluorescein conjugate of the anti-product antibody. The three different media used during the incubation were: (1) cell culture medium, RPMI, 5% fetal calf serum (FCS); (2) RPMI, 5% FCS, supplemented with 40% bovine serum albumin (BSA)(Fluka, Switzerland); and (3) RPMI, 5% FCS, supplemented with 20% BSA and 20% gelatin (Type B from bovine skin approx. 225 bloom, Sigma Chemical Co.) as a gelatinous diffusion inhibitor. The results are shown in FIGS. 9, 10, and 11.

FIG. 9a shows the distribution of labeling of the cells incubated in RPMI, 5% FCS (i.e., without a diffusion inhibitor). The entire cell population is shirted towards higher fluorescence, thus no separation in distinct cell populations can be resolved. FIG. 9b shows the distribution of labeling of cells incubated in RPMI, 5% FCS supplemented with 40% BSA. This BSA medium is a high viscosity diffusion inhibitor. Compared to FIG. 9a, it shows that incubation in this medium led to less background labeling. FIG. 9c shows the distribution of labeling of the cells incubated in RPMI, 5% FCS, supplemented with 20% BSA and 20% gelatin. Using this medium two cell populations, secretors and nonsecretors can be identified. Compared to the cells incubated in the other two media as indicated in FIGS. 9a and 9b, the amount of fluorescence on the secretor population is significantly increased.

This example shows that while a viscous medium such as a high BSA medium will decrease capture of secreted product by non-producer cells, incubation during secretion in a gelatinous medium results in significantly increased labeling of the producer cells with a concomitant lowering of capture non-producer cells. This amplification effect on capture allows the labeling of cells producing lower levels of product and/or allows the use of lower affinity antibodies for the capture of the secreted product. The gelatinous medium appears to result in an increased concentration of the product in the vicinity of the secreting cells while not inhibiting the speed of the capture reaction. When gelatinous media with a cutoff limit lower than the molecular weight of the product is used in the medium, the secreted molecules may concentrate in the gap between cell and medium, resulting in higher local concentrations and more efficient labeling of the secreting cells.

Cell Separation Using MACS

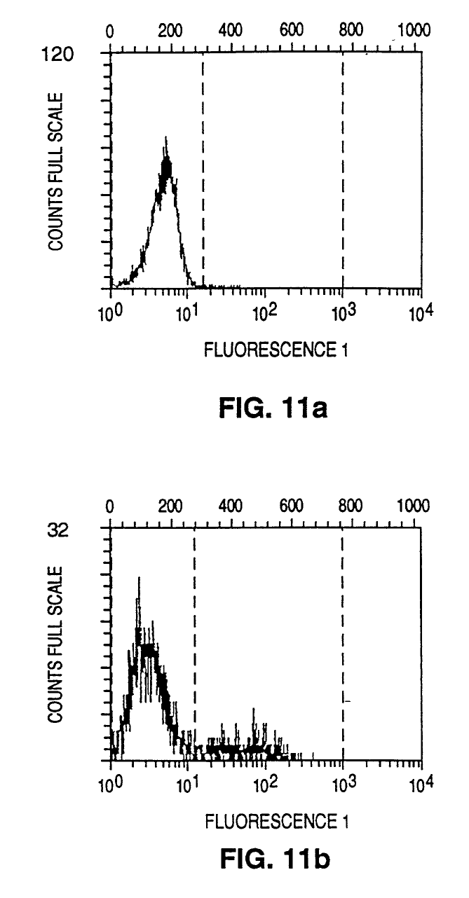

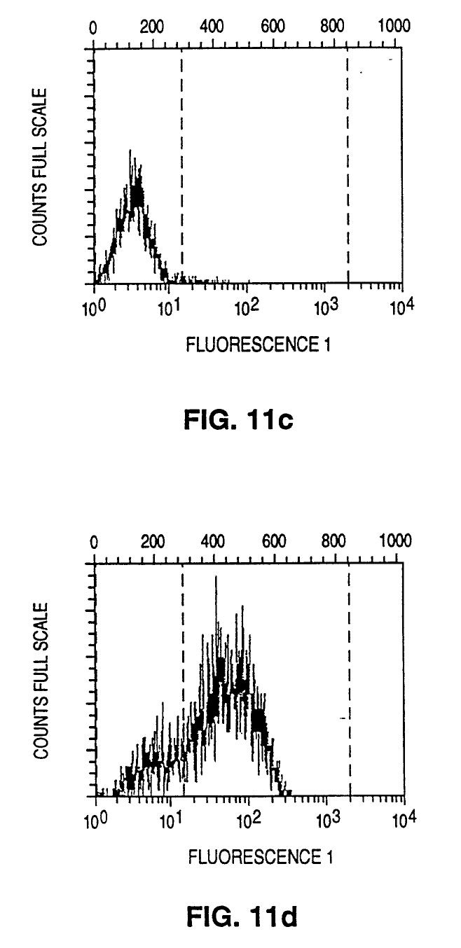

A mixture of B1.8 and X63 cells were chemically biotinylated and labeled with LS136-avidin, as described above. A control sample was taken and stored on ice. The remaining cells were allowed to secrete for 1 hour in 6 ml gelatinous RPMI medium containing 23% gelatin, 18% BSA and 5% FCS. The gel was quickly disoolved in 20 ml of 42° C. PBS, followed by the rapid addition of 30 ml ice-cold PBS and washing in a cooled centrifuge. The cells and the control sample were labeled for 10 minutes on ice with rat anti-mouse IgM microbeads (Miltenyi Biotec GmbH, stained with goat anti-mouse fluorescein (SBA, Birmingham, Ala.) and washed once. The cells were then separated on an A2 column using a MACS magnetic cell sorter. Separation was performed according to the manufacturer's instructions. The control samle, unseparated sample, and magnetic and non-magnetic fractions were analyzed by Flow Cytometry (FACScan)(Becton Dickinson, San Jose, Calif., USA). FIG. 11 a shows the fluorescence distribution of the control sample. As seen in the figure, almost no detectable surface staining was detected on the cells (0.6% in area between dotted lines (positive window)). FIG. 11b shows the fluorescence distribution after secretion and fluorescent labeling, prior to magnetic separation. Approximately 14.2% of the cells are in the positive window and are putative secretors. FIG. 11c shows the fluorescence distribution of the non-magnetic fraction after magnetic separation. Nearly all positive cells are retained in the magnetic column (2% of the cells in positive window). FIG. 11d shows the fluorescence distribution of the magnetic fraction. The population of positive cells is highly enriched (80.3% in positive window). It should be noted that the purity of the cell population can be expected to be higher than shown in the FACScan analysis because of instrument limitations. The enrichment rate can be calculated to greater than 24.

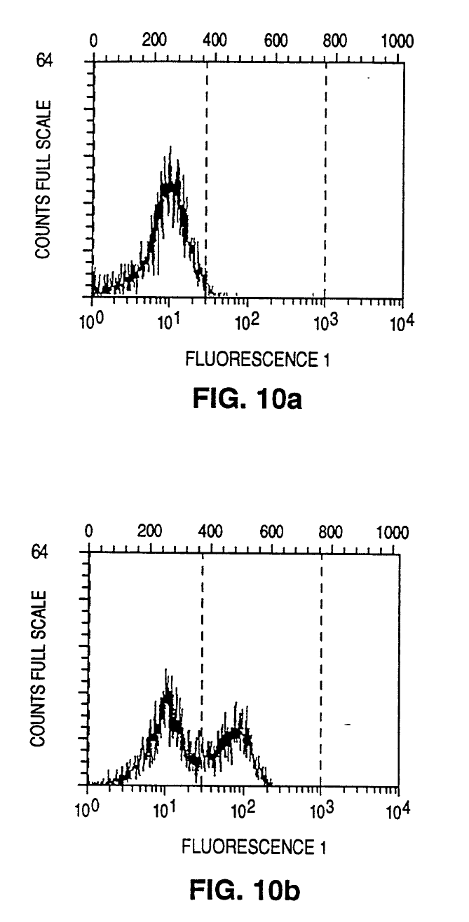

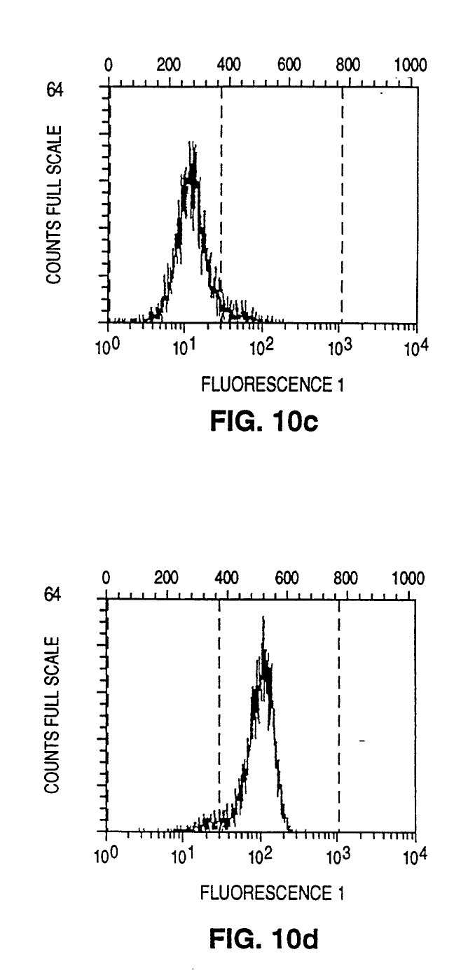

FIGS. 10a to 10d show a similar experiment as in FIG. 11a to FIG. 11d, except that a higher proportion of B1.8 to x63 cells was used. Medium during the secretion phase was RPMI containing 25% gelatin and 2.5% FCS. The percentage of cells in the positive window was 1.3% (control), 41.2% (after secretion), 6.6% (non-magnetic fraction), and 92.9% (magnetic fraction). The enrichment rate for positive cells in this example can be calculated to be greater than 18.7, and the depletion rate greater than 9.9.

### Example 4

The following describes a method to measure the absolute amount of secretion and to compensate for different amounts of capture moiety on the cell surfaces.

During the secretion phase the cells are exposed to a low concentration of tagged product supplied with the medium; the tagged product binds to but does not saturate the product binding sites on the cells. Incubation during this phase causes both the secreted product and the tagged product bind to the cells. The cells are then subjected to labelling with a label specific for the product (both tagged and secreted). Measurement of the tag using one parameter, and the total product in the other parameter, the amount secreted by a cell is normalized, and the different amounts of capture antibody on the cells in the mixture is compensated for.

Commercial Utility

The above described methods and compositions are useful for the detection and/or separation of cells that secrete varying levels of one or more designated substances. The cells may be phenotypically identical except for their secretory activity of the designated product. Thus, the method may be of use in separating cells that secrete commercially valuable substances from those that do not, for example, cells that secrete immunogenic polypeptides, growth factors, molecules that can act as hormones, and a variety of other products, including those produced by recombinant techniques. In addition the techniques may be useful in the isolation of cell groups that are destined for transplantation or implantation procedures, or for packaging for implantation. Illustrative of this type of cell group are the islets of Langerhans, where it would be desirable to segrate groups of cells that are capable of secreting insulin from those that are non-secretors. The methods of determining the distribution of secretory activity of cells in cell mixtures are also of use in large scale fermentations in that they quickly identify the appearance of nonsecretory or low secretory cell variants or of cells producing a modified product.

1. A method to separate cells according to a product secreted and released by said cells, which method comprises effecting a separation of cells according to the degree to which they are labeled with said product,
wherein labeling with said product is achieved by coupling the surface of said cells to a specific binding partner for said product and culturing the cells under conditions wherein said product is secreted, released and specifically bound to said specific binding partner, and wherein the labeled cells are not lysed as part of the separation procedure.

2. The method of claim 1 wherein said product-labeled cells are further labeled with a fluorescent moiety and said separation is conducted by cell sorting.

3. The method of claim 1 wherein said product-labeled cells are further labeled with a magnetic moiety and said separation is conducted in a source of magnetic field.

4. The method of claim 1 wherein said specific binding partner is an antibody or an immunologically reactive fragment thereof.

5. The method of claim 1 wherein said coupling is through a lipid anchor attached to the specific binding partner optionally through a linking moiety.

6. The method of claim 1 wherein said coupling is through an antibody or immunologically reactive fragment thereof attached to said specific binding partner, optionally through a linker.

7. A method to label cells with a product secreted and released by said cells, which method comprises coupling the surface of said cells to a specific binding partner for said product, and culturing the cells under conditions wherein the product is secreted and released.

8. The method of claim 7 wherein said specific binding partner is an antibody or an immunologically reactive fragment thereof.

9. The method of claim 7 wherein said coupling is through a lipid anchor attached to the specific binding partner optionally through a linking moiety.

10. The method of claim 7 wherein said coupling is through an antibody or immunologically reactive fragment thereof attached to said specific binding partner optionally through a linker.

11. A composition of matter which comprises cells capable of capturing a product secreted and released by said cells wherein the surface of said cells is coupled to a specific binding partner for said product.

12. The cells of claim 11 which are further coupled to said product.

13. The cells of claim 11 wherein said specific binding partner is an antibody or an immunologically reactive fragment thereof.

14. The cells of claim 11 wherein said coupling is through a lipid anchor attached to the specific binding partner optionally through a linking moiety.

15. The cells of claim 11 wherein said coupling is through an antibody or immunologically reactive fragment thereof attached to said specific binding partner, optionally through a linker.

16. Cells and progeny thereof separated by the method of claim 1.

17. A method of analyzing a population of cells to determine the proportion of cells that secrete a varying amount of product relative to other cells in the population, the method comprising labeling the cells by the method of claim 7, further labeling the cells with a second label that does not label the captured product, and detecting the amount of product label relative to the second cell label.

18. A method of determining a distribution of secretory activity in a population of cells, the method comprising labeling cells by the method of claim 7, and determining the amount of product label per cell.

19. A method of determining a distribution of product type and secretory activity for the product type in a population of cells, the method comprising labeling cells according to the method of claim 7, wherein the method comprises coupling the surfaces of cells in the population with specific binding partners for each product to be detected, culturing the cells under conditions wherein the products are secreted and released, labeling the secreted capture products, wherein the label for each secreted capture product is distinguishable, and determining the amount and type of product per cell determining the amount of product label per cell.

20. A kit for use in the dectection of cells that secrete a desired product, the kit comprising: a material for use in preparing gelatinous cell culture medium, said medium to be used for cell incubation for the production of the desired secreted product; a product capture system comprised of anchor and capture moieties; a label for detecting the captured product; and instructions for use of the reagents, all packaged in appropriate containers.
